(19) United States
(12) Patent Application Publication
(10) Pub. No.: US 2012/0253068 A1
(43) Pub. Date: Oct. 4, 2012

(54) PROCESS FOR PRODUCING METHIONINE

(76) Inventors: Naoko IMADA, Niihama-shi (JP); Yoshiyuki KOIZUMI, Niihama-shi (JP)

(21) Appl. No.: 13/431,327

(22) Filed: Mar. 27, 2012

(30) Foreign Application Priority Data
Mar. 28, 2011 (JP) 2011-070777

(51) Int. Cl. C07C 319/20

(52) U.S. Cl. 562/559

(57) ABSTRACT

Provided is a process for producing methionine, which involves:
(1) hydrolyzing 5-[2-(methylthio)ethylimidazolidine-2,4-dione in the presence of a basic potassium compound, (2) introducing carbon dioxide into a reaction liquid obtained in step (1), thereby precipitating methionine, and separating the resulting slurry into a precipitate and a mother liquid, (3) heat-treating the mother liquid obtained in step (2), and (4) introducing carbon dioxide into the mother liquid heat-treated in step (3), thereby precipitating methionine and potassium bicarbonate, and separating the resulting slurry into a precipitate and a mother liquid, wherein the alanine content in the mother liquid to be subjected to step (4) is 0.75 wt % or less.

### BACKGROUND OF THE INVENTION

1. Field of the Invention

The present application claims the Paris Convention priority based on Japanese Patent Application No. 2011-070777 filed on Mar. 28, 2011, the entire content of which is incorporated herein by reference.

The present invention relates to a process for producing methionine by a hydrolysis reaction of 5-(2-(methylthio)ethyl)imidazolidine-2,4-dione [see the following reaction formula (1)]. Methionine is useful as a feed additive for animals.

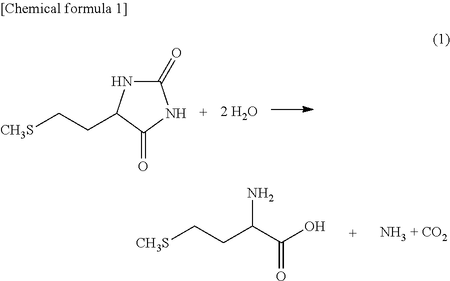

2. Description of the Related Art

As one of processes for producing methionine, there is known a process of hydrolyzing 5-(2-(methylthio)ethyl)imidazolidine-2,4-dione under a basic condition using a basic potassium compound such as potassium carbonate or potassium bicarbonate. In this process, methionine can be obtained through separation as a crystal by introducing carbon dioxide into a reaction liquid after hydrolysis to perform crystallization. However, methionine proportional to the solubility remains in a mother liquid after the separation of methionine, and recyclable potassium bicarbonate is contained therein as the basic potassium compound. For this reason, upon recycling this mother liquid to the hydrolysis reaction, it is necessary to perform purging to a predetermined ratio since impurities are accumulated when the whole quantity is recycled. Since treatment of this purged mother liquid as waste water leads to loss of methionine and potassium bicarbonate contained therein and the waste water treatment is burdensome, this is not advantageous.

Then, various methods of recovering methionine and potassium bicarbonate from the mother liquid as a so-called second crystal have been reported. For example, JP-B-54-9174 discloses mixing the mother liquid with a water-soluble solvent, for example, an alcohol such as methyl alcohol, or acetone, and introducing carbon dioxide into the mixed liquid to perform crystallization. In addition, JP-A-51-1415 discloses concentrating the mother liquid and introducing carbon dioxide into the concentrated liquid to perform crystallization. Further, JP-A-5-320124 discloses mixing the mother liquid with isopropyl alcohol and introducing carbon dioxide into the mixed liquid to perform crystallization. Further, JP-A-2007-63141 discloses concentrating the mother liquid after separation of a first crystal, heat-treating the liquid at 165° C., thereafter, mixing the liquid with isopropyl alcohol, and introducing carbon dioxide into the liquid to perform crystallization.

### SUMMARY OF THE INVENTION

In the above-mentioned methods, the recovery rate of methionine as a second crystal from a mother liquid after separation of a first crystal was not satisfactory.

An object of the present invention is to provide a production process which can improve the recovery rate of methionine as a second crystal.

The present inventors intensively studied and, as a result, found that in a first crystal mother liquid to be subjected to crystallization of a second crystal, alanine generated by degradation of methionine is present as impurities in a relatively large amount and, unexpectedly, this produces a great influence on the recovery rate of methionine as a second crystal. Based on this finding, the present inventors found that the recovery rate of methionine as a second crystal is improved by performing crystallization of a second crystal for a first crystal mother liquid having an alanine content which has been reduced to a specific amount or less, resulting in completion of the present invention.

Further, the present inventors also found that the hydrolysis temperature and the temperature for heat-treating a first crystal mother liquid greatly influence on the alanine content in the first crystal mother liquid to be subjected to crystallization of a second crystal. Based on this finding, the present inventors also found that the alanine content in a first crystal mother liquid to be subjected to crystallization of a second crystal can be reduced to a specific amount or less by precisely controlling the hydrolysis temperature and the temperature for heat-treating a first crystal mother liquid to specific temperatures or lower.

That is, the present invention provides:

[1] A process for producing methionine, comprising the following steps (1) to (4):

(1) a hydrolysis step: a step of hydrolyzing 5-[2-(methylthio)ethylimidazolidine-2,4-dione in the presence of a basic potassium compound,

(2) a first crystallization step: a step of introducing carbon dioxide into a reaction liquid obtained in step (1), thereby precipitating methionine, and separating the resulting slurry into a precipitate and a mother liquid,

(3) a heating step: a step of heat-treating the mother liquid obtained in step (2), and

(4) a second crystallization step: a step of introducing carbon dioxide into the mother liquid heat-treated in step (3), thereby precipitating methionine and potassium bicarbonate, and separating the resulting slurry into a precipitate and a mother liquid, wherein

the alanine content in the mother liquid to be subjected to step (4) is 0.75 wt % or less;

[2] The process according to [1], wherein hydrolysis is carried out at 180.0° C. or lower in step (1), and the mother liquid obtained in step (2) is heat-treated at 180.0° C. or lower in step (3); and

[3] The process according to [1], comprising the step of concentrating at least part of the mother liquid obtained in step (4) and recycling the concentrate to step (3).

According to the present invention, since the hydrolysis temperature in the hydrolysis step (1) and the temperature for heat-treating a first crystal mother liquid in the heating step (3) are precisely controlled, the alanine content in the first crystal mother liquid to be subjected to the second crystallization step (4) is reduced to 0.75 wt % or less. Consequently, methionine which is rapid in growth of a crystal and has a uniform nature (a crystal having a shape closer to a sphere) can be crystallized from the mother liquid, and the recovery rate of methionine as a second crystal can be improved.

### BRIEF DESCRIPTION OF THE DRAWING

FIG. 1 is a drawing showing the reaction flow of Example 1.

### DETAILED DESCRIPTION OF THE INVENTION

In the present invention, 5-[2-(methylthio)ethyl]imidazolidine-2,4-dione is used as a raw material. By hydrolyzing this compound in the presence of a basic potassium compound, a reaction liquid containing methionine as a potassium salt is obtained [hydrolysis step (1)]. The raw material 5-[2-(methylthio)ethyl]imidazolidine-2,4-dione can be prepared, for example, by reacting 2-hydroxy-4-methylthiobutanenitrile with ammonia and carbon dioxide, or ammonium carbonate [see the following reaction formula (2) or (3)].

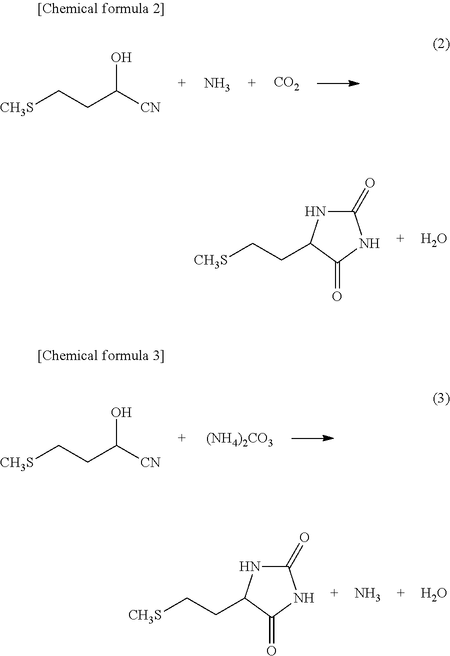

Examples of the basic potassium compound include potassium hydroxide, potassium carbonate, and potassium bicarbonate, and two or more kinds of them can also be used as necessary. The amount of use of the basic potassium compound is usually 2 to 10 equivalents, preferably 3 to 6 equivalents in terms of potassium based on 1 equivalent of 5-[2-(methylthio)ethyl]imidazolidine-2,4-dione. In addition, the amount of use of water is usually 2 to 20-fold by weight based on the amount of 5-[2-(methylthio)ethyl]imidazolidine-2,4-dione.

The hydrolysis reaction is performed in a non-stirring-type, continuous-type reaction tank usually at a temperature of 120° C. or higher, preferably at 173° C. or higher under the pressure of around 0.5 to 1 MPa as expressed by a gauge pressure. From the viewpoint that production of alanine as a byproduct (due to degradation of methionine) can be reduced, the reaction is preferably performed at a temperature of 180.0° C. or lower, more preferably at 179.8° C. or lower. By performing the hydrolysis reaction at a temperature within the above-mentioned range, the alanine content in a first crystal mother liquid to be subjected to the second crystallization step (4) described later can be reduced.

The reaction temperature of hydrolysis is controlled in the order of 10⁻¹° C. or less. The temperature measurement is performed by measuring the temperature of a liquid flowing into a reaction tank for hydrolysis with a temperature measuring device which can precisely measure the temperature in the order of 10⁻¹° C. or less. Since a liquid flowing into the reaction tank is controlled to a desired temperature in the order of 10⁻¹° C. or less by adjusting the steam amount in another reaction tank in advance, and the liquid flows into the reaction tank for hydrolysis in a sufficiently stirred state, the temperature thereof is uniform. In addition, since heating is not performed after the liquid has flowed into the reaction tank for hydrolysis, hydrolysis is not performed at a temperature higher than the measured temperature.

The reaction time depends on the hydrolysis temperature, and is usually 10 minutes to 24 hours, preferably 20 minutes to 2 hours. When the heat treatment time is too short, hydrolysis is insufficient and, conversely, when the heat treatment time is too long, thermal degradation of methionine (production of alanine as a byprOduct, or the like) may occur, and corrosion may occur in the reactor or the like.

In order to take methionine out of the thus obtained hydrolysis reaction liquid, carbon dioxide is introduced into the reaction liquid to perform crystallization, and the resulting slurry is separated into a precipitate and a mother liquid by filtration or decantation, thereby, precipitated methionine is obtained as a first crystal [first crystallization step (2)].

Carbon dioxide is absorbed into the reaction liquid by introduction of carbon dioxide, and a potassium salt of methionine turns into free methionine and is precipitated.

Introduction of carbon dioxide may be performed usually under the pressure of 0.1 to 1 MPa, preferably under 0.2 to 0.5 MPa as expressed by a gauge pressure.

The crystallization temperature is usually 0 to 50° C., preferably 10 to 30° C. As the crystallization time, the time until the hydrolysis reaction liquid is saturated with carbon dioxide and methionine is sufficiently precipitated can be regarded as a guide, and the crystallization time is usually 30 minutes to 24 hours.

Separated methionine may be prepared into a product by performing washing and pH adjustment, and then drying. The drying may be performed by heating to around 50 to 120° C. under a slightly reduced pressure, and the drying time is usually 10 minutes to 24 hours.

Methionine proportional to the solubility remains in a mother liquid after separation of methionine (hereinafter, this mother liquid is referred to as a “first crystal mother liquid”), and potassium bicarbonate which is recyclable as the basic potassium compound is contained therein. For this reason, it is desirable that the first crystal mother liquid is recycled to the hydrolysis reaction in the hydrolysis step (1). Meanwhile, since impurities in the raw material and impurities derived from a side reaction upon hydrolysis, for example, amino acids other than methionine such as glycine and alanine, and coloring components are also contained, these impurities are brought into the hydrolysis reaction by recycling. Then, it is necessary that recycling of the first crystal mother liquid is performed not for the whole quantity but to such a degree that impurities are not accumulated, and the ratio thereof is usually 50 to 90 wt %, preferably 70 to 90 wt % based on the whole quantity of the first crystal mother liquid.

It is desirable that recycling of the first crystal mother liquid is performed by concentrating the mother liquid and using this concentrated liquid as a recycled liquid. By this concentration, carbon dioxide can be distilled off the first crystal mother liquid, and a recycled liquid advantageous for a hydrolysis reaction with enhanced basicity can be obtained. In addition, by performing this concentration at a high temperature of 100 to 140° C., a reaction of converting potassium bicarbonate in the first crystal mother liquid into potassium carbonate (2 KHCO₃K₂CO₃+H₂O+CO₂) is promoted, and a recycled liquid advantageous for a hydrolysis reaction with further enhanced basicity can be obtained. This concentration can be performed under normal pressure, reduced pressure or increased pressure, but in order to perform the concentration at a high temperature as described above, it is effective to adopt the increased pressure condition. The concentration ratio is usually 1.2 to 4-fold, preferably 1.5 to 3.5·fold. Herein, the concentration ratio means the ratio of the liquid weight before concentration relative to the liquid weight after concentration (liquid weight before concentration/liquid weight after concentration), and this is also the same hereinafter.

The first crystal mother liquid after concentration is classified into the mother liquid for recycling and the mother liquid for second crystallization, but the whole quantity can be subjected to second crystallization.

Regarding the first crystal mother liquid for second crystallization, in order to further recover methionine and potassium bicarbonate as a second crystal, the first crystal mother liquid is heat-treated [heating step (3)].

By the heat treatment, methionine dipeptide contained in the first crystal mother liquid is degraded into methionine.

It is preferable that the heating step is performed after addition of a basic potassium compound and, thereby, methionine dipeptide can be effectively degraded into methionine since the heat treatment is performed in the state where the potassium concentration in the mother liquid is high.

Examples of the basic potassium compound include potassium carbonate, potassium bicarbonate, and potassium hydroxide. Among them, potassium hydroxide is preferable.

The amount of addition of the basic potassium compound depends on the methionine dipeptide concentration in the mother liquid, and is preferably 0.25 part by weight or more in terms of potassium based on 100 parts by weight of the mother liquid. The amount of addition is more preferably 0.25 part by weight or more in the case of potassium hydroxide, 1.5 parts by weight or more in the case of potassium carbonate, and 1.0 part by weight or more in the case of potassium bicarbonate. Here, it is preferable that the amount of addition of the basic potassium compound does not exceed 30 parts by weight from the viewpoint that the crystallization efficiency of methionine in the second crystallization step (4) described later is good, and of economy.

The potassium concentration (in terms of potassium) in the mother liquid after addition of the basic potassium compound depends on the methionine dipeptide concentration in the mother liquid, and is preferably 30 wt % or less, particularly preferably 20 wt % or less from the viewpoint that the crystallization efficiency of methionine in the second crystallization step (4) described later is good, and of economy. The lower limit of the potassium concentration is preferably 0.5 wt % or more from the viewpoint of effective degradation of methionine dipeptide. In the present invention, the potassium concentration is measured by ion exchange chromatography (absolute calibration curve method).

The heating step is performed in a non-stirring-type, continuous-type reaction tank. The heating temperature in the heating step depends on the methionine dipeptide concentration in the mother liquid, and the heating is performed usually at a temperature of 150° C. or higher, preferably at 170° C. or higher under the pressure of around 0.5 to 2 MPa as expressed by a gauge pressure. The heating is preferably performed at a temperature of 180.0° C. or lower, more preferably at 179.0° C. or lower from the viewpoint that production of alanine as a byproduct (due to degradation of methionine) can be reduced. By performing the heat treatment at a temperature within the above-mentioned range, the alanine content in the first crystal mother liquid to be subjected to the second crystallization step (4) described later can be reduced.

The heat treatment temperature is controlled in the order of 10⁻¹° C. or less. The temperature measurement is performed by measuring the temperature of a liquid flowing into a reaction tank for heat treatment with a temperature measuring device which can precisely measure the temperature in the order of 10⁻¹° C. or less. Since a liquid flowing into the reaction tank is controlled to a desired temperature with a heat exchanger in the order of 10⁻¹° C. or less in advance, and the liquid flows into the reaction tank for heat treatment in the state of being sufficiently stirred, the temperature thereof is uniform. In addition, since heating is not performed after the liquid has flowed into the reaction tank for heat treatment, the heat treatment is not performed at a temperature higher than the measured temperature.

The heat treatment time depends on the heating temperature, and is preferably 0.3 to 10 hours, more preferably 1 to 3 hours. When the heat treatment time is too short, degradation of methionine dipeptide is delayed and, conversely, when the heat treatment time is too long, thermal degradation of methionine (production of alanine as a byproduct, or the like) occurs, and corrosion may occur in the reactor or the like.

The heat treatment can be performed until the methionine dipeptide content relative to methionine becomes preferably 5 to 30 wt %, more preferably 5 to 18 wt %.

Carbon dioxide is introduced into the first crystal mother liquid after the heat treatment to perform crystallization, and the resulting slurry is separated into a precipitate and a mother liquid by filtration or decantation, thereby, precipitated methionine and potassium bicarbonate are recovered as a second crystal [second crystallization step (4)].

In the first crystal mother liquid to be subjected to the second crystallization step (4), since the alanine content has been reduced to 0.75 wt % or less, preferably 0.60 wt % or less, methionine which is rapid in growth of a crystal and has a uniform nature (a crystal having a shape closer to a sphere) can be crystallized from the mother liquid, and the recovery rate of methionine as a second crystal can be improved.

Such a first crystal mother liquid with the alanine content reduced to 0.75 wt % or less can be obtained, for example, by performing hydrolysis in the hydrolysis step (1) at a temperature of 180.0° C. or lower (preferably 173 to 179.8° C.) and performing the heating step (3) at a temperature of 180.0° C. or lower (preferably 170 to 179.0° C.).

In the first crystal mother liquid after the heat treatment, basicity therein has been risen, and free methionine which has been converted in the first crystallization step returns to a potassium salt of methionine. Therefore, also in the second crystallization step, by introducing carbon dioxide after the heat treatment, a potassium salt of methionine is converted into free methionine again.

The introduction of carbon dioxide can be usually performed under the pressure of 0.1 to 1 MPa, preferably under 0.2 to 0.5 MPa as expressed by a gauge pressure, like the first crystallization step.

The crystallization temperature is usually 0 to 50° C., preferably 5 to 30° C. As the crystallization time, the time until the liquid after the heat treatment is saturated with carbon dioxide and methionine and potassium bicarbonate are sufficiently precipitated can be regarded as a guide, and the crystallization time is usually 10 minutes to 24 hours.

In order to enhance the crystallization efficiency, it is preferable to mix the liquid with a lower alcohol after introduction of carbon dioxide. As the lower alcohol, usually, an alkyl alcohol having an alkyl group with 1 to 5 carbon atoms is used. Above all, alcohols which are miscible with water at an arbitrary ratio, such as methyl alcohol, ethyl alcohol, n-propyl alcohol, isopropyl alcohol, and t-butyl alcohol are preferable, and isopropyl alcohol is particularly preferable. The amount of use of the lower alcohol is usually 0.05 to 5-fold by weight, preferably 0.1 to 2-fold by weight based on the first crystal mother liquid to be subjected to crystallization. In addition, mixing of the first crystal mother liquid and a lower alcohol may be performed before introduction of carbon dioxide, or may be performed simultaneously with introduction of carbon dioxide.

The recovered second crystal (a mixture of methionine and potassium bicarbonate) is preferably recycled to the hydrolysis reaction in the hydrolysis step (1). Thereupon, recycling the second crystal by dissolving the second crystal in the first crystal mother liquid for recycling is preferable in view of operability.

The mother liquid after separation of the second crystal (hereinafter, this mother liquid is referred to as a “second crystal mother liquid”) still contains methionine and potassium bicarbonate. Then, in the present invention, in order to further recover methionine and potassium bicarbonate from this second crystal mother liquid, methionine and potassium bicarbonate are recycled to the heating step (3) after concentration of the second crystal mother liquid to recover them as a third crystal.

By concentration of the second crystal mother liquid, the recovery rate of methionine can be enhanced. This concentration can be performed under the same condition as that of concentration of the first crystal mother liquid to be recycled.

The above-mentioned concentration may be performed on the whole quantity of the second crystal mother liquid for recycling to the heating step (3), or may be performed on part thereof for recycling to the heating step (3).

The above-mentioned steps (1) to (4) may be all performed by a continuous system, or partially performed by a batch system with at least steps (1) and (3) performed by a continuous system.

### EXAMPLES

Hereinafter, examples of the present invention will be described, but the present invention is not limited to the examples. In the examples, “%” and “part(s)” indicating a concentration or an amount of use are on a weight basis unless otherwise indicated.

Here, the methionine recovery rate (%) in Examples 1 to 3 was obtained from the following equation.

Methionine recovery rate (%)=[methionine amount recovered as second crystal/methionine content in inflow liquid to be subjected to second crystallization step]×100

When the recovery rate of methionine is 50% or more (preferably 55% or more), it can be determined that methionine was efficiently recovered.

The concentration of alanine in the examples was measured using LC analysis (an IS method by a fluorescence reaction with OPA) under the following condition.

Apparatus: liquid chromatography mass spectrometer (manufactured by SHINIADZU)
Column temperature: 40° C.
UV absorption wavelength: 340 nm
Flow rate: 1.04 ml/min
Column: SUMIPAX•ODS A-202 (5 μm×6 mmφ×15 cm)
Carrier liquid: MeOH:water=58:42 (15 ml of THF is contained in MeOH), the pH is adjusted with sodium acetate and 40% phosphoric acid.
Injection amount: 1 μL

### Example 1

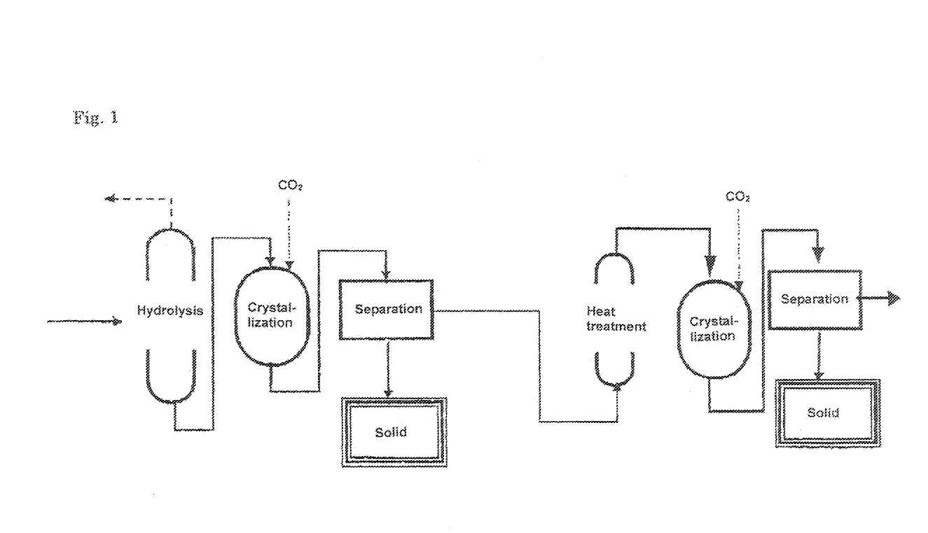

To a reaction tower was continuously supplied a liquid (the whole quantity is 100 parts by weight) containing 7.7 parts by weight of 5-[2-(methylthio)ethyl]imidazolidine-2,4-dione, 8.0 parts by weight of potassium (in terms of simple substance), 3.0 parts by weight of methionine, 0.9 part by weight of methionine dipeptide, and 0.14 part by weight of alanine so that the retention time became 15 minutes. Hydrolysis was performed without stirring while degasification was performed at a constant rate, and a liquid was continuously extracted. The hydrolysis was performed at a gauge pressure of 0.93 MPa and 180.00° C. The degasification was performed so that the extracted liquid amount became 75% of the liquid amount before hydrolysis. By introducing carbon dioxide into a reaction liquid continuously extracted from the hydrolysis tank at a gauge pressure of 0.35. MPa and 20° C., methionine was precipitated, and the resulting slurry was separated into a precipitate and a mother liquid. The resulting mother liquid was concentrated 1.7-fold by heating (135° C.), the resulting concentrated liquid was heated to 180.00° C. with a horizontal-type multitubular heater, and continuously supplied to a drum so that the retention time became 1.2 hours, to heat-degrade methionine dipeptide at a gauge pressure of 1.40 MPa without stirring. When the reaction liquid continuously extracted from the heat degradation tank was concentrated 1.4-fold by heating (135° C.), the alanine content in the resulting concentrated liquid was 0.74 wt %. When carbon dioxide was introduced into the mother liquid at a gauge pressure of 0.30 MPa and 12° C., thereby, methionine and potassium bicarbonate were precipitated, and the resulting slurry was separated into a precipitate and a mother liquid, the methionine recovery rate was 51%. The reaction flow is shown in FIG. 1.

### Example 2

The same treatment as that of Example 1 was performed except that the hydrolysis temperature was 179.80° C., and the temperature for heat-treating the first crystal mother liquid was 179.00° C. in Example 1. The alanine content in the first crystal mother liquid after the heat treatment was 0.58 wt %. The methionine recovery rate was 59%.

### Comparative Example

The same treatment as that of Example 1 was performed except that the hydrolysis temperature was 180.20° C., and the heat degrading temperature of methionine dipeptide was 180.50° C. in Example 1. The alanine content in the first crystal mother liquid after the heat treatment was 1.05 wt %, and the methionine recovery rate was 48%.

TABLE 1

| | | Heat | | |
|---|---|---|---|---|
| | Hydrolysis | treatment | Alanine | Methionine |
| | temperature | temperature | concentration | recovery rate |
| | (° C.) | (° C.) | (wt %) | (%) |
| Example 1 | 180.00 | 180.00 | 0.74 | 51 |
| Example 2 | 179.80 | 179.00 | 0.58 | 59 |
| Comparative | 180.20 | 180.50 | 1.05 | 48 |
| Example | | | | |

According to the present invention, since the process is performed by precisely controlling the hydrolysis temperature in the hydrolysis step (1) and the heat treatment temperature of the first crystal mother liquid in the heating step (3) to specific temperatures or lower, the alanine content in the first crystal mother liquid to be subjected to the second crystallization step (4) is reduced to 0.75 wt % or less, thereby, methionine which is rapid in growth of a crystal and has a uniform nature (a crystal having a shape closer to a sphere) can be crystallized from the mother liquid and, therefore, the recovery rate of methionine as a second crystal can be improved.

What is claimed is:

1. A process for producing methionine, comprising the following steps (1) to (4):
(1) a hydrolysis step: a step of hydrolyzing 5-[2-(methylthio)ethyl]imidazolidine-2,4-dione in the presence of a basic potassium compound,
(2) a first crystallization step: a step of introducing carbon dioxide into a reaction liquid obtained in step (1), thereby precipitating methionine, and separating the resulting slurry into a precipitate and a mother liquid,
(3) a heating step: a step of heat-treating the mother liquid obtained in step (2), and
(4) a second crystallization step: a step of introducing carbon dioxide into the mother liquid heat-treated in step (3), thereby precipitating methionine and potassium bicarbonate, and separating the resulting slurry into a precipitate and a mother liquid, wherein
the alanine content in the mother liquid to be subjected to step (4) is 0.75 wt % or less.

2. The process according to claim 1, wherein hydrolysis is carried out at 180.0° C. or lower in step (1), and the mother liquid obtained in step (2) is heat-treated at 180.0° C. or lower in step (3).

3. The process according to claim 1, comprising the step of concentrating at least part of the mother liquid obtained in step (4) and recycling the concentrate to step (3).
